(19) United States
(12) Patent Application Publication
(10) Pub. No.: US 2023/0138246 A1
(43) Pub. Date: May 4, 2023

(54) USE OF DIHYDROBERBERINE OR ITS DERIVATIVES FOR ANTI-AGING

(71) Applicant: NANJING NUTRABUILDING BIO-TECH CO., LTD., Nanjing (CN)

(72) Inventors: Ronghua YI, Nanjing (CN); Mingru WANG, Nanjing (CN); Shawn WELLS, Lewisville, TX (US); Kylin LIAO, Nanjing (CN)

(21) Appl. No.: 17/975,028

(22) Filed: Oct. 27, 2022

(30) Foreign Application Priority Data
Nov. 2, 2021 (CN) PCT/CN2021/128232

(51) Int. Cl. A61K 8/49; A61Q 19/08; A23L 33/10

(52) U.S. Cl. CPC A61K 8/49; A61Q 19/08; A23L 33/10; A23V 2002/00

(57) ABSTRACT

The present invention provides novel methods for anti-aging, especially ameliorating or preventing skin aging, cellular senescence, or photoaging through inhibiting progerin in a mammal. The method includes administration to the mammal a composition including an effective amount of dihydroberberine or a pharmaceutically acceptable salt, acid, ester, analog or derivative thereof. The present invention also provides a composition including an effective amount of dihydroberberine or a pharmaceutically acceptable salt, acid, ester, analog or derivative thereof, for anti-aging, especially ameliorating or preventing skin aging, cellular senescence, or photoaging through inhibiting progerin in a mammal.

### CROSS REFERENCE TO RELATED APPLICATION

This application claims the benefit of a PCT International Application Number PCT/CN2021/128232, filed on Nov. 2, 2021, the content of which is hereby incorporated by reference in its entirety for all purposes.

### FIELD OF THE INVENTION

The present invention generally relates to compositions and methods for anti-aging, especially ameliorating or preventing skin aging, cellular senescence, or photoaging in a mammal, in particular through inhibiting progerin.

### BACKGROUND OF THE INVENTION

Progerin is a truncated version of the lamin A protein. Progerin is most often generated by a single nucleotide polymorphism (C1824T) in the gene that codes for Lamin A. This mutation activates a cryptic splice site and gives rise to a form of lamin A with a deletion of 50 amino acids near the C-terminus. Lamin A constitutes a major structural component of the lamina, a scaffold of proteins found inside the nuclear membrane of a cell. Progerin protein aberrantly accumulates in the nuclear membrane, preventing at least some of the normal scaffolding functions of lamin A, which in turn interferes with multiple processes in the nucleus, including chromatin organization, heterochromatin formation, the DNA-damage response, cell cycle, gene transcription, and telomere maintenance. Progerin interacts with cell environment and causes changes in the location and level of chromatin remodeling factors, transcription factors, DNA repairing factors, factors connected with the nuclear lamina. All of these changes influence cell hyperproliferation and cause an arrest of the cell, apoptosis, resulting in cellular senescence, dysfunction of tissue and organs.

Progerin-dependent mechanisms act in natural aging (Ashapkin, V. V., et al., Are There Common Mechanisms Between the Hutchinson-Gilford Progeria Syndrome and Natural Aging? Frontiers in genetics, 2019. 10: p. 455). Besides, ultraviolet radiation (UVR) induces progerin expression, which contributes to photoaging of the skin. (Takeuchi, H. and T. M. Rünger, Longwave UV light induces the aging-associated progerin. The Journal of investigative dermatology, 2013. 133(7): p. 1857-1862).

Cellular senescence is one of the most important in vivo mechanisms related to aging. Senescent cells impair tissue function by irreparable cell damage resulting from natural aging, consequently restricting the lifespan. The replicative senescence, a major reason of cellular senescence, seen after approximately sixty rounds of cell division in cultures (Hayflick's limit), results from the progressive erosion of telomeres following each division. This progressive erosion leads to telomere dysfunction and irreversible cell cycle arrest.

Compositions and methods are needed against the negative effects of progerin on aging and especially skin aging, cellular senescence, or photoaging.

### SUMMARY OF THE PRESENT INVENTION

In a first aspect, the present invention provides a method for anti-aging in a mammal, the method comprising administration to the mammal a composition comprising an effective amount of dihydroberberine or a pharmaceutically acceptable salt, acid, ester, analog or derivative thereof.

In some embodiments, the anti-aging is achieved through inhibiting progerin.

In some embodiments, the anti-aging includes mitigating or preventing skin aging, cellular senescence, or photoaging.

In some embodiments, the mammal is human.

In some embodiments, the composition is prepared in a form of food, drink, nutritional supplement, cosmetic product, or pharmaceutical composition.

In some embodiments, the dihydroberberine or a pharmaceutically acceptable salt, acid, ester, analog or derivative thereof is administrated at a daily dose of 2-2000 mg. In some embodiments, the dihydroberberine or a pharmaceutically acceptable salt, acid, ester, analog or derivative thereof is administrated at a daily dose of 5-1500 mg, 20-1000 mg, 40-800 mg, 80-600 mg, or 100-400 mg. In some embodiments, the daily dose is administered in divided doses or a single dose.

In some embodiments, the administration is through various routes selected from oral administration, intravenous injection, intramuscular injection, intraperitoneal injection, topical application, or sublingual application. In some embodiments, the administration is at least once a day or more times a day.

In some embodiments, the dihydroberberine or a pharmaceutically acceptable salt, acid, ester, analog or derivative thereof is formulated in solutions, liquid suspensions, parenteral solutions, injections, tablets, pills, granules, powders, film, (micro)capsules, aerosols, tonics, or syrups, beverages, nourishments, snacks, bars, gums, sugars, a facial mask composition, a functionalized cream composition, a functionalized essence, a skin care composition, a make-up composition or a functionalized food composition.

In a second aspect, the present invention provides a composition comprising an effective amount of dihydroberberine or a pharmaceutically acceptable salt, acid, ester, analog or derivative thereof, for anti-aging in a mammal.

In some embodiments, the anti-aging is achieved through inhibiting progerin.

In some embodiments, the anti-aging includes mitigating or preventing skin aging, cellular senescence, or photoaging.

In some embodiments, the composition is prepared in a form of food, drink, nutritional supplement, cosmetic product, or pharmaceutical composition.

In some embodiments, the dihydroberberine or a pharmaceutically acceptable salt, acid, ester, analog or derivative thereof is administrated at a daily dose of 2-2000 mg. In some embodiments, the dihydroberberine or a pharmaceutically acceptable salt, acid, ester, analog or derivative thereof is administrated at a daily dose of 5-1500 mg, 20-1000 mg, 40-800 mg, 80-600 mg, or 100-400 mg. In some embodiments, the daily dose is administered in divided doses or a single dose.

In some embodiments, the mammal is human. In some embodiments, the administration is through various routes selected from oral administration, intravenous injection, intramuscular injection, intraperitoneal injection, topical application, or sublingual application. In some embodiments, the administration is at least once a day or more times a day.

In some embodiments, the dihydroberberine or a pharmaceutically acceptable salt, acid, ester, analog or derivative thereof is formulated in solutions, liquid suspensions, parenteral solutions, injections, tablets, pills, granules, powders, film, (micro)capsules, aerosols, tonics, or syrups, beverages, nourishments, snacks, bars, gums, sugars, a facial mask composition, a functionalized cream composition, a functionalized essence, a skin care composition, a make-up composition or a functionalized food composition.

In a third aspect, the present invention provides use of a composition for preparing food, drink, nutritional supplement, cosmetic product, or pharmaceutical composition for anti-aging in a mammal, wherein the composition comprises an effective amount of dihydroberberine or a pharmaceutically acceptable salt, acid, ester, analog or derivative thereof.

In some embodiments, the anti-aging is achieved through inhibiting progerin.

In some embodiments, the anti-aging includes mitigating or preventing skin aging, cellular senescence, or photoaging.

In some embodiments, the dihydroberberine or a pharmaceutically acceptable salt, acid, ester, analog or derivative thereof is administrated at a daily dose of 2-2000 mg. In some embodiments, the dihydroberberine or a pharmaceutically acceptable salt, acid, ester, analog or derivative thereof is administrated at a daily dose of 5-1500 mg, 20-1000 mg, 40-800 mg, 80-600 mg, or 100-400 mg. In some embodiments, the daily dose is administered in divided doses or a single dose.

In some embodiments, the mammal is human. In some embodiments, the administration is through various routes selected from oral administration, intravenous injection, intramuscular injection, intraperitoneal injection, topical application, or sublingual application. In some embodiments, the administration is at least once a day or more times a day.

In some embodiments, the dihydroberberine or a pharmaceutically acceptable salt, acid, ester, analog or derivative thereof is formulated in solutions, liquid suspensions, parenteral solutions, injections, tablets, pills, granules, powders, film, (micro)capsules, aerosols, tonics, or syrups, beverages, nourishments, snacks, bars, gums, sugars, a facial mask composition, a functionalized cream composition, a functionalized essence, a skin care composition, a make-up composition or a functionalized food composition.

In a fourth aspect, the present invention provides a method for inhibiting progerin in a mammal, the method comprising administration to the mammal a composition comprising an effective amount of dihydroberberine or a pharmaceutically acceptable salt, acid, ester, analog or derivative thereof.

In some embodiments, the method is used for anti-aging.

In some embodiments, the anti-aging includes mitigating or preventing skin aging, cellular senescence, or photoaging.

In some embodiments, the mammal is human.

In some embodiments, the composition is prepared in a form of food, drink, nutritional supplement, cosmetic product, or pharmaceutical composition.

In some embodiments, the dihydroberberine or a pharmaceutically acceptable salt, acid, ester, analog or derivative thereof is administrated at a daily dose of 2-2000 mg. In some embodiments, the dihydroberberine or a pharmaceutically acceptable salt, acid, ester, analog or derivative thereof is administrated at a daily dose of 5-1500 mg, 20-1000 mg, 40-800 mg, 80-600 mg, or 100-400 mg. In some embodiments, the daily dose is administered in divided doses or a single dose.

In some embodiments, the administration is through various routes selected from oral administration, intravenous injection, intramuscular injection, intraperitoneal injection, topical application, or sublingual application. In some embodiments, the administration is at least once a day or more times a day.

In some embodiments, the dihydroberberine or a pharmaceutically acceptable salt, acid, ester, analog or derivative thereof is formulated in solutions, liquid suspensions, parenteral solutions, injections, tablets, pills, granules, powders, film, (micro)capsules, aerosols, tonics, or syrups, beverages, nourishments, snacks, bars, gums, sugars, a facial mask composition, a functionalized cream composition, a functionalized essence, a skin care composition, a make-up composition or a functionalized food composition.

In a fifth aspect, the present invention provides a composition comprising an effective amount of dihydroberberine or a pharmaceutically acceptable salt, acid, ester, analog or derivative thereof, for inhibiting progerin in a mammal.

In some embodiments, the composition may be used for anti-aging.

In some embodiments, the anti-aging includes mitigating or preventing skin aging, cellular senescence, or photoaging.

In some embodiments, the composition is prepared in a form of food, drink, nutritional supplement, cosmetic product, or pharmaceutical composition.

In some embodiments, the dihydroberberine or a pharmaceutically acceptable salt, acid, ester, analog or derivative thereof is administrated at a daily dose of 2-2000 mg. In some embodiments, the dihydroberberine or a pharmaceutically acceptable salt, acid, ester, analog or derivative thereof is administrated at a daily dose of 5-1500 mg, 20-1000 mg, 40-800 mg, 80-600 mg, or 100-400 mg. In some embodiments, the daily dose is administered in divided doses or a single dose.

In some embodiments, the mammal is human. In some embodiments, the administration is through various routes selected from oral administration, intravenous injection, intramuscular injection, intraperitoneal injection, topical application, or sublingual application. In some embodiments, the administration is at least once a day or more times a day.

In some embodiments, the dihydroberberine or a pharmaceutically acceptable salt, acid, ester, analog or derivative thereof is formulated in solutions, liquid suspensions, parenteral solutions, injections, tablets, pills, granules, powders, film, (micro)capsules, aerosols, tonics, or syrups, beverages, nourishments, snacks, bars, gums, sugars, a facial mask composition, a functionalized cream composition, a functionalized essence, a skin care composition, a make-up composition or a functionalized food composition.

In a sixth aspect, the present invention provides use of a composition for preparing food, drink, nutritional supplement, cosmetic product, or pharmaceutical composition for inhibiting progerin in a mammal, wherein the composition comprises an effective amount of dihydroberberine or a pharmaceutically acceptable salt, acid, ester, analog or derivative thereof.

In some embodiments, the composition may be used for anti-aging.

In some embodiments, the anti-aging includes mitigating or preventing skin aging, cellular senescence, or photoaging.

In some embodiments, the dihydroberberine or a pharmaceutically acceptable salt, acid, ester, analog or derivative thereof is administrated at a daily dose of 2-2000 mg. In some embodiments, the dihydroberberine or a pharmaceutically acceptable salt, acid, ester, analog or derivative thereof is administrated at a daily dose of 5-1500 mg, 20-1000 mg, 40-800 mg, 80-600 mg, or 100-400 mg. In some embodiments, the daily dose is administered in divided doses or a single dose.

In some embodiments, the mammal is human. In some embodiments, the administration is through various routes selected from oral administration, intravenous injection, intramuscular injection, intraperitoneal injection, topical application, or sublingual application. In some embodiments, the administration is at least once a day or more times a day.

In some embodiments, the dihydroberberine or a pharmaceutically acceptable salt, acid, ester, analog or derivative thereof is formulated in solutions, liquid suspensions, parenteral solutions, injections, tablets, pills, granules, powders, film, (micro)capsules, aerosols, tonics, or syrups, beverages, nourishments, snacks, bars, gums, sugars, a facial mask composition, a functionalized cream composition, a functionalized essence, a skin care composition, a make-up composition or a functionalized food composition.

In a seventh aspect, the present invention provides a method for preparing a composition disclosed herein.

These and other features, aspects, and advantages of the present invention will become better understood with reference to the following description and appended claims.

### BRIEF DESCRIPTION OF THE DRAWINGS

FIG. 1a is a graph of lamin A and progerin mRNA expression levels in AG01972 cells supplemented with different concentrations of DHB.

FIG. 1b is a graph of lamin A and progerin mRNA expression levels in 2DD cells supplemented with different concentrations of DHB.

FIG. 2a is a graph of protein levels of lamin A and progerin in AG01972 cells of DHB supplemented group and non-supplemented group.

FIG. 2b is a graph of protein levels of lamin A and progerin in 2DD cells of DHB supplemented group and non-supplemented group.

FIG. 3a is a graph of effect of DHB at different concentrations on the proliferation of PD45 2BS cells and WI38 cells measured by MTT-assay.

FIG. 3b is a graph of time course of the effect of DHB on proliferation of 2BS cells.

FIG. 3c is a graph of time course of the effect of DHB on proliferation of WI38 cells.

FIG. 3d is a graph of quantification of SA-β-gal-positive cell rate of 2BS cells.

### DETAILED DESCRIPTION OF THE INVENTION

In the Summary Section above and the Detailed Description Section, and the claims below, reference is made to particular features of the invention. It is to be understood that the disclosure of the invention in this specification includes all possible combinations of such particular features. For example, where a particular feature is disclosed in the context of a particular aspect or embodiment of the invention, or a particular claim, that feature can also be used, to the extent possible, in combination with and/or in the context of other particular aspects and embodiments of the invention, and in the invention generally.

Skin aging is a complex process. Macroscopically, skin aging is recognized by fine wrinkles, loss of elasticity, orange-peel or dull appearance of the skin, reduced epidermal and dermal thickness. Microscopically, typical characteristics of skin aging include epidermal atrophy, decreased mitotic rate of basal keratinocytes, decreased proliferative capacity and cellular senescence, atrophy of the dermal extracellular matrix and change of the physiological properties of connective tissues.

Berberine is a bioactive compound that can be extracted from several different plants, including a group of shrubs called Berberis. Berberine is most commonly taken for diabetes, high levels of cholesterol or other fats (lipids) in the blood (hyperlipidemia), and high blood pressure. It is also used for burns, canker sores, liver disease, and many other conditions. However, there is no report about the relationship between berberine and progerin, between berberine and anti-aging and/or ameliorating or preventing skin aging and/or ameliorating or preventing cellular senescence and/or ameliorating or preventing photoaging. Serendipitously, the inventors have discovered that berberine and metabolites thereof can suppress the expression of progerin, thus resisting aging, particularly skin aging, cellular senescence, and/or photoaging.

Dihydroberberine (DHB) is a metabolite of berberine. Pharmacokinetic analyses have indicated that nitroreductases of the gut microbiota reduces BBR to its absorbable form Dihydroberberine (DHB), which displays improved absorption and enhanced oral bioavailability (Feng, R., et al., Transforming berberine into its intestine-absorbable form by the gut microbiota. 2015. 5(1): p. 1-15).

As used herein, the term “or” is meant to include both “and” and “or.” In other words, the term “or” may also be replaced with “and/or.”

As used herein, the singular forms “a,” “an” and “the” are intended to include the plural forms as well, unless the context clearly indicates otherwise.

As used herein, the term “comprise” or “include” and their conjugations, refer to a situation wherein said terms are used in their non-limiting sense to mean that items following the word are included, but items not specifically mentioned are not excluded. It also encompasses the more limiting verb ‘to consist essentially of’ and ‘to consist of’.

As used herein, the term “effective amount” refers to the amount required to achieve the effect as taught herein. The specific effective dose level for any particular subject will depend upon a variety of factors including the signs being treated and the severity of the signs; the specific composition employed; the age, body weight, general health, sex and diet of the subject; the time of administration, route of administration, and rate of excretion of dihydroberberine (DHB) or its analog or its derivatives employed; the duration of the treatment; and like factors well known in the medical arts. For example, it is well known within the skill of the art to start doses of the compound at levels lower than those required to achieve the desired effect and to gradually increase the dosage until the desired effect is achieved.

One of skill in the art recognizes that an amount may be considered “effective” even if the condition is not totally eradicated or prevented, but it or its symptoms and/or effects are improved or alleviated partially in the subject.

As used herein, the term “pharmaceutically acceptable” means pharmaceutically, physiologically, alimentarily, and/or nutritionally acceptable, and refers to those compositions or combinations of agents, materials, or compositions, and/or their dosage forms, which are within the scope of sound medical judgment, suitable for use in contact with the tissues of human beings and animals without excessive toxicity, irritation, allergic response, or other problem or complication, commensurate with a reasonable benefit/risk ratio.

As used herein, the term “mammal” or “subject” may be used interchangeably to refer to any animal to which the presently disclosed methods and compositions may be applied or administered. The animal may have an illness or other disease, but the animal does not need to be sick to benefit from the presently disclosed methods and compositions. As such any animal may apply the disclosed combinations, compositions or kits, or be a recipient of the disclosed methods. “Mammal” includes, without limitation, mice, rats, rabbits, guinea pigs, dogs, cats, sheep, goats, cows, horses, primates, such as monkeys, chimpanzees, and apes, and, in particular, humans. Although the animal subject is preferably a human, the methods and compositions of the invention have application in veterinary medicine.

The dosage of dihydroberberine or a pharmaceutically acceptable salt, acid, ester, analog or derivative thereof and/or composition comprising the same may range broadly, depending upon the desired effects and the indication. The daily dosage regimen for an adult human patient may be, for example, an oral dose of between 0.01 mg and 3000 mg of dihydroberberine or its analog or derivative, preferably between 1 mg and 700 mg, e.g., 5 to 200 mg, or between about 0.1 mg and about 1,000 mg of dihydroberberine or its analog or derivative per kg of body weight of the subject. The dosage may be a single one or a series of two or more given in the course of one or more days, as is needed by the subject. In some embodiments, the compounds are administered for a period of time, for example for a week or more, or for months or years.

As used herein, the term “administration” refers to the process of delivering a disclosed composition or active ingredient to a subject. The compositions of the invention can be administered in a variety of ways, including orally, intragastrically, and parenterally (e.g., intravenous and intraarterial as well as other suitable parenteral routes), and the like.

As used herein, a “parenteral solution” refers to a solution that can be administered elsewhere in the body than the mouth and alimentary canal. It is not delivered via the intestinal tract. For example, parenteral solution can be delivered intravenously.

As used herein, a “tonic” refers to a medicinal substance taken to give a feeling of vigor or well-being.

As used herein, a “syrup” refers to a thick sticky liquid derived from a sugar-rich plant, for example, sugar cane, corn, and maple.

Multiple techniques of administering a composition exist in the art including, but not limited to, oral, rectal, topical, aerosol, injection and parenteral delivery, including intramuscular, subcutaneous, intravenous, intramedullary injections, intrathecal, direct intraventricular, intraperitoneal, intranasal and intraocular injections.

“Intraperitoneal” as used here means within or administered through the peritoneum. The peritoneum is a thin, transparent membrane that lines the walls of the abdominal (peritoneal) cavity and contains/encloses the abdominal organs such as the stomach and intestines.

As used herein, “sublingual” refers to situated or applied under the tongue.

A “functionalized cream composition” includes a cream composition that has a potentially positive effect on health beyond basic nutrition.

An “essence” includes an extract or concentrate obtained from a particular plant or other matter and used for flavoring or scent. A “functionalized essence” includes an essence that has a potentially positive effect on health beyond basic nutrition.

A “functionalized food composition” includes a food composition that has a potentially positive effect on health beyond basic nutrition.

Any titles or subheadings used herein are for organization purposes and should not be used to limit the scope of embodiments disclosed herein.

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for.

All publications and patent applications cited in this specification are herein incorporated by reference as if each individual publication or patent application were specifically and individually indicated to be incorporated by reference.

### EXAMPLES

### Example 1

This example describes cell culture procedure, and treatments of composition according to some embodiments and their resulted gene expression changes.

The AG01972 primary HGPS cell line was obtained from Coriell Cell Repositories (New Jersey, USA). Human dermal fibroblasts (HDF) were isolated from juvenile foreskins and termed 2DD was also used. AG01972 cells were cultured in T75 tissue culture treated flasks (Fisher, UK) containing Dulbecco's Minimum Essential Media+Glutamax (Invitrogen, UK), supplemented with 15% fetal calf serum and 1% penicillin/streptomycin (Invitrogen) in an humidified, 37° C., 5% CO₂/95% air incubator. The cells were passaged twice weekly. Cells were plated at a density of 2×10⁵ into 9 cm dishes containing 13 mm diameter glass coverslips. Six hours after seeding, the AG01972 cells were treated with 0.1% dimethyl sulfoxide, different concentrations of DHB. DHB treatment was repeated 24 h after the initial treatment. The cells were analyzed after 48 h of treatment.

Total RNA was isolated using a RNeasy Micro extraction kit according to the manufacturer's protocol. An on-column DNase I digestion was performed to avoid genomic DNA amplification. The RNA level and quality were checked using the Nanodrop technology. A total of 500 ng of RNA was used for reverse transcription using the Superscript III reverse transcription kit. The quantitative PCR analysis was performed following the manufacturers' instructions.

Quantification of gene expression was based on the DeltaCt Method and normalized on 18S expression. The PCR primers have been described previously by S. Rodriguez and colleagues (Rodriguez, S., et al., Increased expression of the Hutchinson-Gilford progeria syndrome truncated lamin A transcript during cell aging. 2009. 17(7): p. 928-937). The primer sequences for lamin A (exons 11/12), 5′-TCTTCTGCCTCCAGTGTCACG-3′ (SEQ ID NO: 1) and 5′-AGTTCTGGGGGCTCTGGGT-3′ (SEQ ID NO: 2), progerin (exons 11/12), 5′-ACTGCAGCAGCTCGGGG-3′ (SEQ ID NO: 5) and 5′-TCTGGGGGCTCTGGGC-3′ (SEQ ID NO: 6). Taqman MGB probe sequences for lamin A (exon 11), 5′-ACTCGCAGCTACCG-3′ (SEQ ID NO: 7), progerin (exon 11), 5′-CGCTGAGTACAACCT-3′ (SEQ ID NO: 9). Reporter and quencher dyes for the LMNA locus assays are 5′-6FAM and 3′-non-fluorescent quencher dye. 18s (Assay HS_99999901_s1) probes and the primers were provided by Life Technologies. 18S ribosomal RNA (abbreviated 18S rRNA) is a part of the ribosomal RNA. 18S rRNA is the structural RNA for the small component of eukaryotic cytoplasmic ribosomes, and thus one of the basic components of all eukaryotic cells.

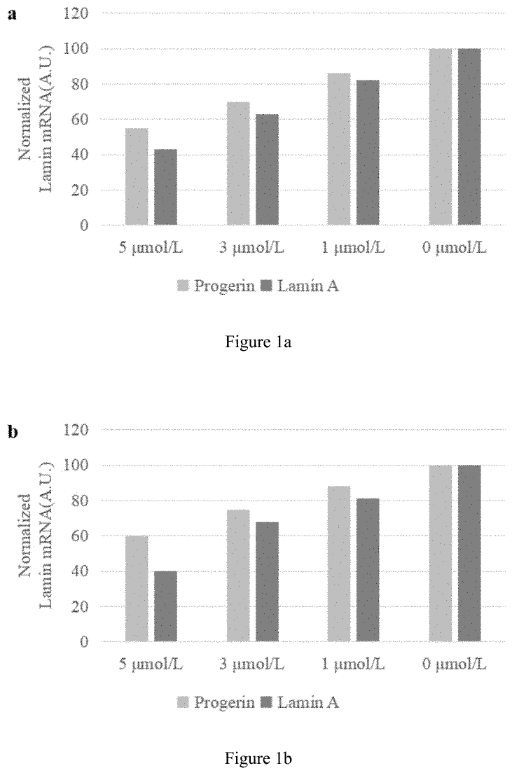

The protein levels of A-type lamins and mRNA expressions of lamin A and progerin were measured 48 h after DHB treatment on AG01972 cells. FIG. 1a is a graph of lamin A and progerin mRNA expression levels in AG01972 cells supplemented with different concentrations of DHB. FIG. 1a shows a correlated dose-dependent decrease in lamin A and progerin expression compared with untreated cells, with a maximum effect at 5 μmol/L. All further experiments presented in this study were performed at this dose. The effect of treatment with 5 μmol/L DHB on lamin A expression was then confirmed in 2DD cells, showing a decrease in lamin A mRNA expression. FIG. 1b is a graph of lamin A and progerin mRNA expression levels in 2DD cells supplemented with different concentrations of DHB.

### Example 2

This example describes treatments of composition according to some embodiments inhibit the expression level of Progerin protein.

Whole-cell lysates of AG01972 cells and 2DD cells were collected, separated by SDS polyacrylamide gel electrophoresis, and transferred onto polyvinylidene fluoride membrane using the liquid transfer method. The blots were blocked in 10% skim milk in Tween 0.1% tris-buffered saline 1×1 h at room temperature. The membranes were incubated during the night at 4° C. Antigen-antibody binding was detected using horseradish peroxidase-conjugated species-specific secondary antibodies, followed by enhanced chemiluminescence western blotting detection reagents. The western blot results were quantified using ImageJ software.

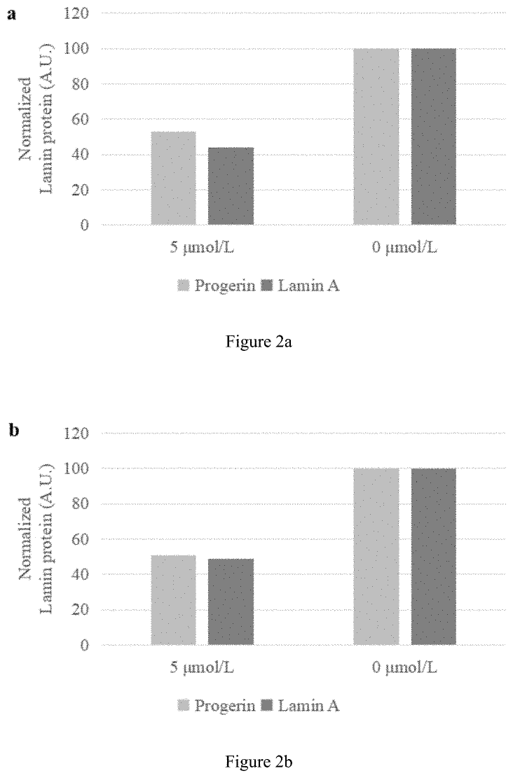

FIG. 2a is a graph of protein levels of lamin A and progerin in AG01972 cells of DHB supplemented group and non-supplemented group. FIG. 2b is a graph of protein levels of lamin A and progerin in 2DD cells of DHB supplemented group and non-supplemented group. Western blot analysis confirmed the significant decrease in progerin content in AG01972 cells, as well as the decrease in lamin A in AG01972 cells and 2DD cells treated with 5 μmol/L DHB compared with untreated cells. This data shows that DHB can inhibit the expression level of lamin A and decrease progerin protein levels.

The studies on AG01972 cells and 2DD cells treated with DHB show that DHB inhibits the expression of lamin A and inhibiting progerin in vitro cell models at protein and mRNA levels, suggesting DHB possesses good potential to be further developed into a promising candidate to anti-aging.

### Example 3

This example describes treatments of composition according to some embodiments extended the replicative lifespan, improved the morphology, and boosted rejuvenation markers of replicative senescence in human fetal lung diploid fibroblasts (2BS and WI38).

Human diploid fibroblast 2BS and WI38 cells were isolated from female fetal lung fibroblast tissue. These cell lines were purchased from commercially available sources. Cells were grown in Dulbecco's Modification Eagle's Minimum Essential Medium (DMEM, Life Technologies Inc.) supplemented with 10% fetal bovine serum (FBS; Gibco), 2 mm glutamine, 100 U/ml penicillin, and 100 μg/ml streptomycin, in a humidified atmosphere with 5% CO₂ at 37° C. These cells were considered as young at PD30 or below and fully senescent at PD50 or above

Cell-cycle analysis: Briefly, WI38 or 2BS cells, grown to approximately 85% confluency at PD45, were split in the ratio of 1:2. One of them was resuspended in DMEM containing different concentrations of DHB (0, 0.300, 0.625, and 2.5 μg/ml) and incubated at 37° C. in 5% CO₂ for 72 hr, while the other one was resuspended in normal DMEM and incubated under similar conditions. The DNA content of the cells was measured by fluorescence-activated cell sorting method using a Becton-Dickinson FAC Scan Flow Cytometry System (BD). The data were analyzed using CellFIT software.

Cell cytotoxicity assay: The CCK-8 assay kit (Dojindo) was used for testing cytotoxicity in vitro. At PD45, 2BS or WI38 cells were seeded into flat-bottomed 96-well microplates at a density of 5×10³ cells/0.2 ml per well. After 20 hr, when the cells reached a subconfluent state, the cells were transferred to a special culture medium containing various concentrations of DHB for further growth, at 37° C. in 5% CO₂ up to 24 hr. Cell viability (%)=(OD_{treatment group}−OD_{blank})/(OD_{control group}−OD_{blank})×100.

Cell proliferation was assayed using the CCK-8 method. Cells were seeded into 96-well plates at 2.5×10³ cells/0.2 ml per well and incubated with different DHB concentrations (0, 0.300, 0.625, and 2.5 μg/ml) for one week. The absorbance values of each well were measured at day 0 (4 h after plating) and on days 1, 2, 3, 4, 5, and 6. The medium containing DHB or DMSO was refreshed every 24 hr. At the indicated points, cells were harvested with 10% CCK-8 for one hour, and the absorbance was measured at 490 nm. Each data point was measured five times, and each curve was repeated more than three times.

Senescence-associated β-galactosidase (SA-β-gal) assay: 2B S cells (PD45) grown in DMEM with/without 0.300 μg/ml DHB concentration were stained according to the method of Dimri with minor modifications (Dimri, Lee et al. 1995). Cells grown on plates were washed with PBS, fixed in 4% formaldehyde for 5 min at room temperature, and washed again with PBS. Then, cells were incubated overnight at 37° C. without CO₂ in a freshly prepared staining buffer (1 mg/ml 5-bromo-4-chloro-3-indolyl-β-d-galactopyranoside (X-gal), 40 mm citric acid/sodium phosphate, pH 6.0, 5 mm potassium ferrocyanide, 5 mm potassium ferricyanide, 150 mm NaCl, and 2 mm MgCl₂) for 16 h before being examined according to the instruction manual (CST, 9860S). The average value of the percentage of SA-β-gal-positive cells for each set of samples was calculated based on three independent experiments.

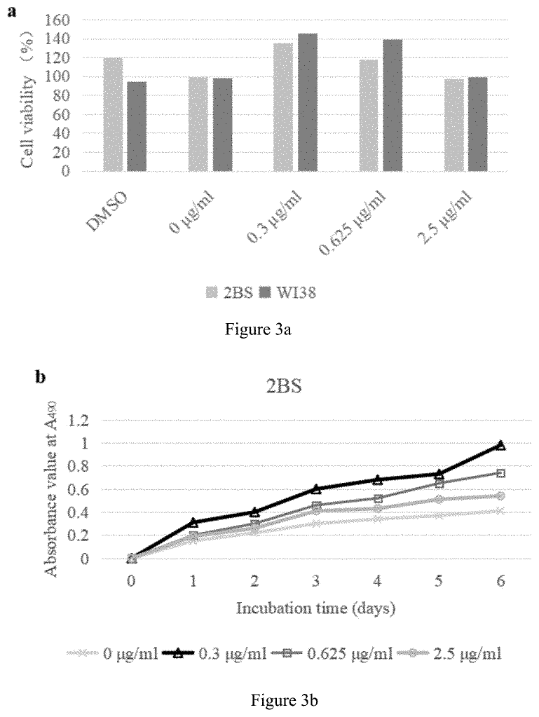

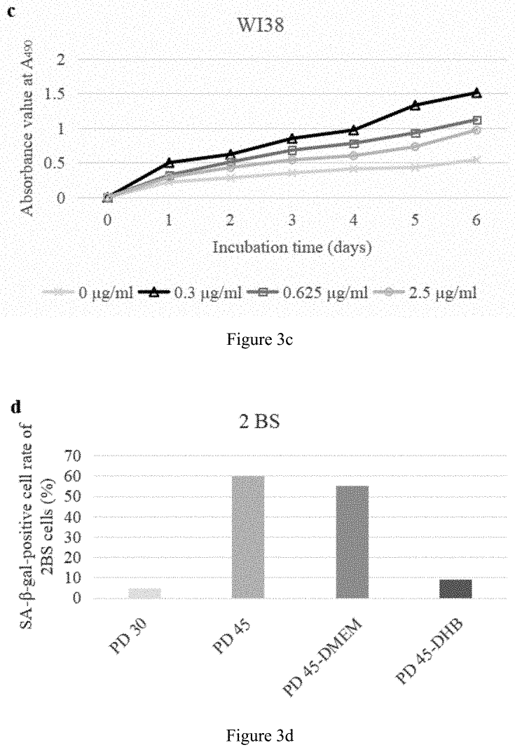

The effects of low concentration DHB on replicative senescence and its mechanism using human fetal lung diploid fibroblast cells (2BS and WI38) was tested. These cell lines are considered as young at PD30 or below and fully senescent at PD50 or above. Cell proliferation was used to evaluate the aging state of the cultured cells. FIG. 3a is a graph of effect of DHB at different concentrations on the proliferation of PD45 2BS cells and WI38 cells measured by MTT-assay. As shown in FIG. 3a, the optimum concentration of DHB was determined to be 0.3 μg/ml. FIG. 3b is a graph of time course of the effect of DHB on proliferation of 2BS cells. FIG. 3c is a graph of time course of the effect of DHB on proliferation of WI38 cells. In addition, DHB, at a concentration of 0.3 μg/ml, promoted proliferation of 2BS (PD45) and WI38 (PD45) cells during seven days of incubation (FIGS. 3b and 3c). Furthermore, the positive rate of cells for SA-β-gal is usually used as a marker of population (Dimri, Lee et al. 1995). FIG. 3d is a graph of quantification of SA-β-gal-positive cell rate of 2BS cells. Quantification analysis of SA-β-gal in FIG. 3d is shown that DHB (about 9%) could delay or reverse the population senescence of 2BS cells remarkably, as compared to the PD 45-DMEM group (about 55%) and PD 45 group (about 60%).

Although specific embodiments and examples of this invention have been illustrated herein, it will be appreciated by those skilled in the art that any modifications and variations can be made without departing from the spirit of the invention. The examples and illustrations above are not intended to limit the scope of this invention. Any combination of embodiments of this invention, along with any obvious their extension or analogs, are within the scope of this invention. Further, it is intended that this invention encompass any arrangement, which is calculated to achieve that same purpose, and all such variations and modifications as fall within the scope of the appended claims.

What is claimed is:

1. A method for anti-aging in a mammal, the method comprising administration to the mammal a composition comprising an effective amount of dihydroberberine or a pharmaceutically acceptable salt, acid, ester, analog or derivative thereof.

2. The method of claim 1, wherein the anti-aging is achieved through inhibiting progerin.

3. The method of claim 1, wherein the anti-aging includes mitigating or preventing skin aging, cellular senescence, or photoaging.

4. The method of claim 1, wherein the mammal is human.

5. The method of claim 1, wherein the composition is prepared in a form of food, drink, nutritional supplement, cosmetic product, or pharmaceutical composition.

6. The method of claim 1, wherein the dihydroberberine or a pharmaceutically acceptable salt, acid, ester, analog or derivative thereof is administrated at a daily dose of 2-2000 mg.

7. The method of claim 1, wherein the administration is through various routes selected from oral administration, intravenous injection, intramuscular injection, intraperitoneal injection, topical application, or sublingual application.

8. The method of claim 1, wherein the dihydroberberine or a pharmaceutically acceptable salt, acid, ester, analog or derivative thereof is formulated in solutions, liquid suspensions, parenteral solutions, injections, tablets, pills, granules, powders, film, (micro)capsules, aerosols, tonics, or syrups, beverages, nourishments, snacks, bars, gums, sugars, a facial mask composition, a functionalized cream composition, a functionalized essence, a skin care composition, a make-up composition or a functionalized food composition.

9. A composition comprising an effective amount of dihydroberberine or a pharmaceutically acceptable salt, acid, ester, analog or derivative thereof, for anti-aging in a mammal.

10. The composition of claim 9, wherein the anti-aging is achieved through inhibiting progerin.

11. The composition of claim 9, wherein the anti-aging includes mitigating or preventing skin aging, cellular senescence, or photoaging.

12. The composition of claim 9, wherein the mammal is human.

13. The composition of claim 9, wherein the composition is prepared in a form of food, drink, nutritional supplement, cosmetic product, or pharmaceutical composition.

14. The composition of claim 9, wherein the dihydroberberine or a pharmaceutically acceptable salt, acid, ester, analog or derivative thereof is administrated at a daily dose of 2-2000 mg.

15. The composition of claim 9, wherein the dihydroberberine or a pharmaceutically acceptable salt, acid, ester, analog or derivative thereof is formulated in solutions, liquid suspensions, parenteral solutions, injections, tablets, pills, granules, powders, film, (micro)capsules, aerosols, tonics, or syrups, beverages, nourishments, snacks, bars, gums, sugars, a facial mask composition, a functionalized cream composition, a functionalized essence, a skin care composition, a make-up composition or a functionalized food composition.

16. Use of a composition for preparing food, drink, nutritional supplement, cosmetic product, or pharmaceutical composition for anti-aging in a mammal, wherein the composition comprises an effective amount of dihydroberberine or a pharmaceutically acceptable salt, acid, ester, analog or derivative thereof.

17. The use of claim 16, wherein the anti-aging is achieved through inhibiting progerin.

18. The use of claim 16, wherein the anti-aging includes mitigating or preventing skin aging, cellular senescence, or photoaging.

19. The use of claim 16, wherein the dihydroberberine or a pharmaceutically acceptable salt, acid, ester, analog or derivative thereof is administrated at a daily dose of 2-2000 mg.

20. The use of claim 16, wherein the dihydroberberine or a pharmaceutically acceptable salt, acid, ester, analog or derivative thereof is formulated in solutions, liquid suspensions, parenteral solutions, injections, tablets, pills, granules, powders, film, (micro)capsules, aerosols, tonics, or syrups, beverages, nourishments, snacks, bars, gums, sugars, a facial mask composition, a functionalized cream composition, a functionalized essence, a skin care composition, a make-up composition or a functionalized food composition.
